(19) United States
(12) Patent Application Publication
(10) Pub. No.: US 2018/0036220 A1
(43) Pub. Date: Feb. 8, 2018

(54) HYALURONIC ACID PARTICLES FOR COSMETIC OR DERMATOLOGICAL USES

(71) Applicant: CENTRE NATIONAL DE LA RECHERCHE SCIENTIFIQUE, Paris (FR)

(72) Inventor: Kawthar BOUCHEMAL, Palaiseau (FR)

(21) Appl. No.: 15/552,318

(22) PCT Filed: Feb. 26, 2016
(86) PCT No.: PCT/FR2016/050447

§ 371 Date: Aug. 21, 2017

(30) Foreign Application Priority Data
Feb. 27, 2015 (FR) 1551711

(51) Int. Cl. A61K 8/73; A61K 9/00; A61K 31/728; A61K 8/02; A61Q 19/08

(52) U.S. Cl. CPC A61K 8/735; A61K 8/0241; A61Q 19/08; A61K 31/728; A61K 9/0014; A61K 2800/30; A61K 2300/00

(57) ABSTRACT

In the field of cosmetics or pharmaceuticals, and particularly to the dermatological field, disclosed are particles containing hyaluronic acid modified by hydrophobic groups and the use thereof as an agent for stimulating the epidermis and/or dermis. In particular, the assemblies are useful on the one hand, as a moisturising and/or plumping and/or volumising agent. On the other hand, the particles containing hydrophobised hyaluronic acid are useful for reactivating endogenous hyaluronic acid expression thereby making it possible to reduce and/or prevent skin imperfections such as filling in wrinkles and lines.

### FIELD OF THE INVENTION

This invention relates to the field of cosmetics or pharmaceuticals, and particularly to the dermatological field. In particular, this invention relates to particles containing hyaluronic acid modified by hydrophobic groups and the use thereof as an agent for stimulating the epidermis and/or dermis. In particular, these assemblies are useful on the one hand, as a moisturising and/or plumping and/or volumising agent. On the other hand, these particles comprising hydrophobised hyaluronic acid are useful for reactivating endogenous hyaluronic acid expression thereby making it possible to reduce and/or prevent skin imperfections such as filling in wrinkles and lines. The particles of the invention are also useful for accelerating the healing of the skin or mucous membranes.

### BACKGROUND OF THE INVENTION

Hyaluronic acid is a polysaccharide of the family of glycosaminoglycans comprised of two repeat units: β-(1,3)-D glucuronic acid and β-(1,4)-N-acetyl-D-glucosamine. This polysaccharide is widely present in the human body, in particular among the connecting, epithelial and nerve tissues. More particularly, on the skin, the production of hyaluronic acid makes it possible to fill in the intercellular spaces and participates strongly in the moisturising, tone and elasticity of the skin as well as in tissue cohesion.

The decrease in the production of hyaluronic acid in the organism starting at the age of 40 years, is one of the explanations of skin ageing. In recent years, this polymer has therefore been widely used in the formulation of cosmetic products to combat the effects of ageing.

Hyaluronic acid of a high molar weight (2,000 to 6,000 kDa) has a very low activity on the epidermis and has no activity on the dermis. Hyaluronic acid of a high molar weight is therefore not able to diffuse, and therefore act, correctly within skin structures.

There is therefore a need to provide anti-ageing formulations wherein the diffusion of hyaluronic acid is facilitated both on the epidermis and on the dermis.

In order to overcome this difficulty, industrialists have developed commercial products wherein the hyaluronic acid has been hydrolysed. However, hyaluronic acid with a low molar weight has a high production cost and studies have shown that using hydrolysed hyaluronic acid on the skin induces inflammation phenomena.

There is therefore a need to provide anti-ageing formulations that resolve the problems described hereinabove. In particular, it is necessary to allow for the diffusion of hyaluronic acid both on the epidermis and on the dermis.

The solution of this invention is to provide the hyaluronic acid in the form of particles. Contrary to native hyaluronic acid of a high molar weight which has a very low activity, the hyaluronic acid in the form of particles of the invention is able to diffuse in the epidermis and in the dermis.

In this invention, the formulation in the form of particles is made possible by using hydrophobised hyaluronic acid, i.e. hyaluronic acid modified by the introduction of hydrophobic groups.

Surprisingly, the Applicant has revealed that such particles of hydrophobised hyaluronic acid makes it possible on the one hand to effectively access the epidermal and dermal structures; and on the other hand, that these particles of hydrophobised hyaluronic acid make it possible to reactivate the endogenous production of hyaluronic acid. These effects are demonstrated hereinafter in the examples.

The particles of hydrophobised hyaluronic acid of the invention are therefore of major interest in producing anti-ageing formulations and in obtaining a moisturising and/or plumping and/or volumising effect of the skin. The particles of the invention also have a healing effect on superficial wounds of the skin and of the mucous membranes.

The Applicant has previously described in WO2013/15019 particles of inclusion complexes of hydrophobised hyaluronic acid and of alpha-cyclodextrin, as encapsulating systems. In particular, the particles of WO2013/150193 are intended to vectorise a substance of interest to a target tissue.

WO2013/150193 does not indicate that any of the particles thereof of inclusion complexes of hydrophobised hyaluronic acid and of alpha-cyclodextrin could diffuse both on the epidermis and on the dermis and as such have an anti-ageing effect.

In WO2013/150193, the presence of cyclodextrin is presented as being essential for the formation of particles containing hydrophobised hyaluronic acid. WO2013/150193 does not describe or suggest particles containing hydrophobised hyaluronic acid, but that do no comprise cyclodextrin. On the contrary, on example 30 of this document, it is indicated that in the absence of cyclodextrin, there is no formation of particles.

Surprisingly, the Applicant has revealed in this invention that particles could however be obtained in the absence of cyclodextrin.

In WO2013/150193, the hydrophobised hyaluronic acid used had a substantial substitution rate with hydrophobic chains. The hydrophobised hyaluronic acid of WO2013/150193 therefore had substantial hydrophobicity.

On the contrary, in this invention, it has been shown that by controlling the general hydrophobicity of the hydrophobised hyaluronic acid, it was possible to obtain particles without having to add cyclodextrin. The controlling of the general hydrophobicity of the hydrophobised hyaluronic acid can be done by adjusting the various following aspects, simultaneously or not:
the molar weight of the hyaluronic acid used;
the substitution rate of the hyaluronic acid with hydrophobic chains;
the nature of the hydrophobic groups used;
the type of bonds at play for bonding the hydrophobic groups to the hyaluronic acid.

For example, modifying a hyaluronic acid of a high molecular weight by introducing alkyl hydrophobic groups via ester bonds, with a low substitution rate, leads to a limited general hydrophobicity, allowing for the forming of particles in the absence of cyclodextrin. On the contrary, when the substitution rate is increased, only insoluble aggregates are obtained and it is not possible to form particles without having to add a cyclodextrin.

The general hydrophobicity of the hydrophobised hyaluronic acid used to form the particles of the invention can be evaluated using infrared spectroscopy. In particular, the intensity of the characteristic absorption bands of the alkyl functions is representative of the hydrophobicity. Intense bands are synonymous with excessive hydrophobicity which does not make it possible to obtain particles, while less intense bands are characteristic of hydrophobised hyaluronic acid that alone can form particles.

### SUMMARY

The invention therefore relates to the use in the cosmetics field of particles containing hyaluronic acid modified by hydrophobic groups or the salts and/or derivatives thereof, having the formula (I):

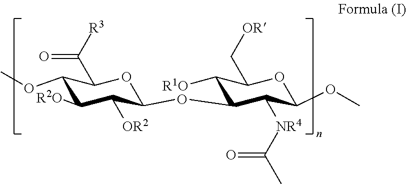

wherein
R¹ and R² each independently represent a hydrogen atom, an R group or a —COR group wherein R represents:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
R³ represents:
   a hydroxyl group;
   a —O⁻X⁺ group wherein X⁺ represents a cation; preferably, a sodium ion; or
   a —OR′ group or a —NHR′ group wherein R′ represents:
      a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
      a linear or branched alkene group, containing 2 to 1,000 carbon atoms and bearing at least one C═C double bond;
R⁴ represents a hydrogen atom or an R″ group chosen from:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group;
with said particles not comprising cyclodextrin.

According to one embodiment, said particles are particles of O-palmitoyl-hyaluronic acid, of O-oleoyl-hyaluronic acid, of O-stearyl-hyaluronic acid or of O-lauryl-hyaluronic acid.

According to one embodiment, the invention relates to the use of said particles as a volumising agent and/or plumping agent.

According to one embodiment, the invention relates to the use of said particles in order to improve the moisturising of the mucous members, of the epidermis and of the dermis.

According to one embodiment, the invention relates to the use of said particles for limiting and/or correcting imperfections of the skin; preferably, as a tensing agent of the skin or as an agent that favours the filling in of wrinkles and/or lines of the skin.

The invention also relates to particles containing hyaluronic acid modified by hydrophobic groups, or the salts and/or derivatives thereof, having the formula (I):

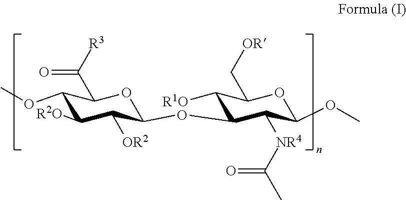

wherein
R¹ and R² each independently represent a hydrogen atom, an R group or a —COR group wherein R represents:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
R³ represents:
   a hydroxyl group;
   a —O⁻X¹ group wherein X¹ represents a cation; preferably, a sodium ion; or
   a —OR′ group or a —NHR′ group wherein R′ represents:
      a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
      a linear or branched alkene group, containing 2 to 1,000 carbon atoms and bearing at least one C═C double bond;
R⁴ represents a hydrogen atom or an R″ group chosen from:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group;
with said particles not comprising cyclodextrin;
for their use as a medicament.

According to one embodiment, the particles comprising hyaluronic acid for their use as a medicament, are used as a dermatological agent; preferably, as a healing agent.

The invention also relates to particles comprising hyaluronic acid modified by hydrophobic groups, or the salts and/or derivatives thereof, having the formula (I), such as defined hereinabove.

According to one embodiment, the particles of the invention comprise hyaluronic acid modified by hydrophobic groups, or the salts and/or derivatives thereof, having the formula (Ia):

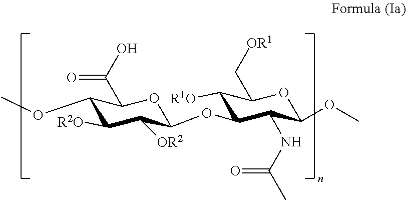

wherein n, R¹ and R² are such as defined in the formula (I) and with the condition that at least one of R¹ or R² represents a —COR group.

The invention also relates to a pharmaceutical composition comprising particles according to the invention and a pharmaceutically acceptable carrier.

The invention further relates to a medical device comprising particles according to the invention.

The invention also has for object the use of a medical device according to the invention for repairing superficial lesions of the skin or of the mucous membranes; preferably for healing superficial wounds of the skin.

### DEFINITIONS

In this invention, the terms hereinbelow are defined in the following way:
“Alkyl”: relates to a linear or branched hydrocarbon chain, comprising from 1 to 1,000 carbon atoms; preferably, from 1 to 60 carbon atoms, more preferably from 1 to 20 carbon atoms, more preferably, methyl, ethyl, propyl, isopropyl, n-butyl, sec-butyl, isobutyl, tert-butyl, pentyl, hexyl, heptyl, octyl, nonyl, decyl, undecyl, dodecyl, tridecyl, tetradecyl, pentadecyl, cetyl, heptadecyl, octadecyl, nonadecyl, eicosyl, oleoyl, linoleyl, linolenyl; even more preferably methyl, ethyl, propyl, isopropyl, n-butyl, sec-butyl, isobutyl, tert-butyl, pentyl, hexyl, heptyl, octyl, nonyl, decyl, undecyl, dodecyl, tridecyl, tetradecyl, pentadecyl, cetyl, heptadecyl, octadecyl, nonadecyl and eicosyl;
“Alkene”: relates to a linear or branched unsaturated hydrocarbon chain, comprising at least 2 carbon atoms, characterised by the presence of at least one double covalent bond between two carbon atoms;
“Cyclodextrin”: relates to a cyclic oligosaccharide comprised of β-D-glucopyranose sub-units bonded by α-(1,4) bonds such as for example alpha-cyclodextrin;
“Palmitoyl”: relates to a —CO—(CH₂)₁₄—CH₃ group,

“Particles”: relates to assemblies comprising hyaluronic acid modified by hydrophobic groups. In this invention, the term “particle” comprises the microparticles and/or the nanoparticles obtained from hydrophobised hyaluronic acid;
“Hydrophobic”: relates to a compound or a chemical group that does not have affinity for water or for aqueous mediums;
“Wrinkle” or “Line”: relates to a fold on the surface of the skin of a more or less substantial depth, caused by a loss of skin tonicity; in particular, by a rupture of the elastic fibres of the dermis with harming of the connecting tissue;
“Moisturising”: relates to the action carried out to prevent drying of the skin; preferably, to counter the drying of the epidermis and/or of the dermis and/or of the mucous membranes, in particular to counter the drying of the epidermis and/or of the dermis;
“Dermis”: relates to the connecting tissue forming the skin with the epidermis and the hypodermis. The dermis is comprised of the papillary dermis, the reticular dermis and the deep dermis;
“Papillary dermis”: relates to the connecting tissue forming the skin comprised of protruding papillae and juxtaposing the epidermis;
“Epidermis”: relates to the superficial layer of the skin of which the surface is formed of dead keratinised cells;
“Plumping agent”: relates to a compound able to generate a measurable increase in the density of the epidermis and/or of the dermis over any zone of the human body. The “plumping” effect of the dermis or of the epidermis can be analysed by the fringe projection technique or by ultrasound scans or by the implementation of techniques suited to the zone of the human body for which it is desired to highlight this effect;
“Volumising agent”: relates to a compound able to generate a measurable increase in the volume of the epidermis and/or of the dermis over any zone of the human body. The “volumising” effect can be analysed, measured and quantified by ultrasound scans or by the implementation of techniques suited to the zone of the human body for which it is desired to highlight this effect;
“Tensing agent”: relates to a compound that can reduce the imperfections of the skin (preferably, of the epidermis and/or of the dermis) or limit the appearance thereof; in particular, able to reduce the wrinkles and/or lines in a subject. The “tensing” effect can be analysed, measured and quantified by ultrasound scans or by the implementation of techniques suited to the zone of the human body for which it is desired to highlight this effect, for example using a Cutometer®;
“Healing agent”: relates to a compound able to favour the healing of wounds, in particular of superficial wounds, in particular superficial wounds of the skin or of the mucous membranes;
“Cosmetic use”: relates to a use intended to embellish and/or improve the appearance of the human body;
“Superficial wound”: relates to a wound reaching only the cutaneous envelope or the immediately underlying tissues, such as but not limited to, a simple localised abrasion of the skin (scratches and scrapes);
“Fatty acid”: relates to carboxylic acid with a saturated or unsaturated carbon aliphatic chain having from 4 to 36 carbon atoms; preferably, a carboxylic acid of which the carbon aliphatic chain has from 4 to 28 atoms; more preferably, from 8 to 20 atoms;
“Pharmaceutically acceptable”: refers to the ingredients of a pharmaceutical composition that are compatible together and not deleterious for the patient.

### DETAILED DESCRIPTION

Particles of Hydrophobised Hyaluronic Acid

The invention has for object particles of hyaluronic acid modified by hydrophobic groups, or the salts and/or derivatives thereof. In particular, the invention relates to particles containing hyaluronic acid modified by hydrophobic groups or the salts and/or derivatives thereof, having the formula (I):

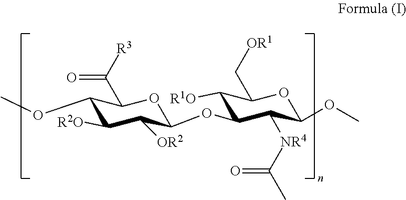

wherein
R¹ and R² each independently represent a hydrogen atom, an R group or a —COR group wherein R represents:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 carbon atoms; or
   a linear or branched alkene group, containing 2 to 1,000 carbon atoms and bearing at least one C═C double bond;
R³ represents:
   a hydroxyl group;
   a —O⁻X¹ group wherein X¹ represents a cation; preferably, a sodium ion; or
   a —OR′ group or a —NHR′ group wherein R′ represents:
      a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
      a linear or branched alkene group, containing 2 to 1,000 carbon atoms and bearing at least one C═C double bond;
R⁴ represents a hydrogen atom or an R″ group chosen from:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 carbon atoms; or
   a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group.

According to one embodiment, the particles of the invention do not comprise cyclodextrin.

According to one embodiment, the particles of the invention are constituted of hyaluronic acid modified by hydrophobic groups or the salts and/or derivatives thereof, having the formula (I).

According to one embodiment, the hyaluronic acid is modified by acylation of the hydroxyl functions by fatty acids and the derivatives thereof; preferably, by fatty acids that have a saturated or unsaturated carbon aliphatic chain that has from 4 to 36 carbon atoms; more preferably, a carbon aliphatic chain that has from 4 to 28 atoms; even more preferably, a carbon aliphatic chain that has from 8 to 20 atoms.

According to one embodiment, the hyaluronic acid is modified by fatty acids and the derivatives thereof; preferably, caprylic acid, capric acid, lauric acid, myristic acid, palmitic acid, stearic acid, arachidic acid, oleic acid, linoleic acid; with this list in no way being complete or limiting.

| Fatty acid | Formula |
|---|---|
| Caprylic acid (C8) | 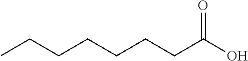 |
| Capric acid (C10) | 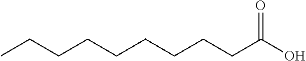 |
| Lauric acid (C12) | 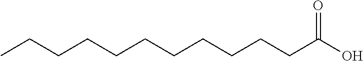 |
| Myristic acid (C14) | 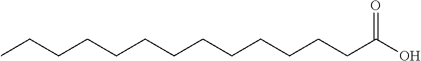 [SMILES: CCCCCCCCCCCCCC(=O)O] |
| Palmitic acid (C16) | 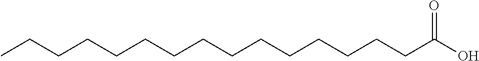 [SMILES: CCCCCCCCCCCCCCCC(=O)O] |
| Stearic acid (C18) | 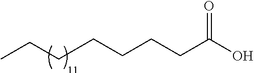 |
| Arachidic acid (C20) | 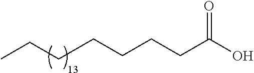 |
| Oleic acid (C18) | 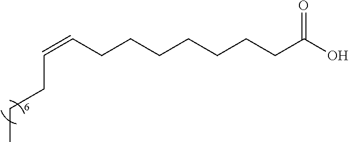 |

According to one embodiment, the hyaluronic acid is modified by the palmitic acid.

According to one embodiment, the hyaluronic acid is modified by the oleic acid.

According to one embodiment, the hyaluronic acid is modified by the lauric acid.

According to one embodiment, the hyaluronic acid is modified by the stearic acid.

According to one embodiment, the preferred compounds of the invention are the compounds of the formula (I) wherein R¹ and R² each independently represent a hydrogen atom, an—R group or a —COR group wherein R represents a linear alkyl group containing 15 or 16 carbon atoms.

According to one embodiment, the modified hyaluronic acid is octyldodecyl erucate hyaluronate.

According to one embodiment, the hyaluronic acid is not modified by an aromatic group.

According to one embodiment, the particles of the invention comprisehyaluronic acid modified by hydrophobic groups or the salts and/or derivatives thereof, having the formula (Ia):

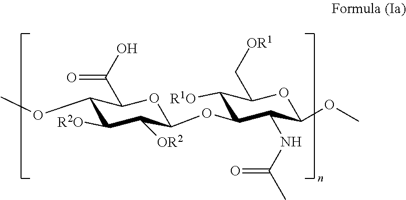

wherein n, R¹ and R² are such as defined in the formula (I) and with the condition that at least one of R¹ or R² represents a —COR group.

According to one embodiment, in the formula (Ia), R represents a linear alkyl group containing from 1 to 20 carbon atoms, preferably from 6 to 20 carbon atoms, more preferably of 7, 9, 11, 13, 15, 17 or 19 carbon atoms.

According to another embodiment, in the formula (Ia), R represents a linear alkene group, containing 2 to 20 carbon atoms and bearing at least one C═C double bond. Preferably, R represents a linear alkene group containing 6 to 20 carbon atoms, more preferably 7, 9, 11, 13, 15, 17 or 19 carbon atoms. Advantageously, R represents a linear alkene group comprising a C═C double bond.

According to one embodiment, the hydrophobic groups are covalently fixed to the hyaluronic acid. According to one embodiment, the hydrophobic groups are covalently fixed to the hyaluronic acid by one or several oxygen atoms of said hyaluronic acid; in particular on the carboxylic function, and/or a hydroxyl function of said hyaluronic acid. According to one embodiment, at least one of the hydroxyl groups of the hyaluronic acid reacts with the fatty acids or the derivatives thereof, such as but not limited to, acid chlorides or acid anhydrides, in order to provide covalent bonds of the ester type. The hydrophobic group is therefore covalently bonded to the hyaluronic acid by one of the oxygen atoms thereof, in the form of an acyl group (O-acylation reaction). According to an embodiment, the palmitic acid is covalently bonded to the hyaluronic acid by at least one of the oxygen atoms thereof, in the form of an acyl group (O-acylation reaction). The acylation reaction is known to those skilled in the art. It is described for example in the international application published under number WO2013/150193.

According to one embodiment, the hydrophobic groups are covalently fixed to the hyaluronic acid by one or several oxygen atoms of said hyaluronic acid in proportions ranging from 0.001% to 100%; preferably, ranging from 0.1% to 70%; more preferably, from 0.5 to 20%; preferably from 0.5 to 20%.

According to one embodiment, the hydrophobic groups are covalently fixed to the hyaluronic acid by one or several nitrogen atoms of said hyaluronic acid; in particular on the amide or carboxylic function of said hyaluronic acid.

According to one embodiment, the hydrophobic groups are covalently fixed to the hyaluronic acid by one or several nitrogen atoms of said hyaluronic acid in proportions ranging from 0.001% to 100%; preferably, ranging from 0.1% to 70%; more preferably, from 0.5 to 20%; preferably from 0.5 to 20%.

According to one embodiment, the hydrophobic groups are covalently fixed to the hyaluronic acid by a bond of the ether type. According to an embodiment, the hydrophobic groups are covalently fixed to the hyaluronic acid by a bond of the ester type. According to one embodiment, the hydrophobic groups are covalently fixed to the hyaluronic acid by a bond of the amide type. According to an embodiment, the hydrophobic groups are covalently fixed to the hyaluronic acid by a bond of the carbamate type.

According to one embodiment, the degree of substitution of the hyaluronic acid by the hydrophobic groups is within a range from 0.001% to 100%; preferably, ranging from 0.1% to 70%; more preferably, from 0.5 to 20%. According to one embodiment, the degree of substitution of the hyaluronic acid by the hydrophobic groups is within a range from 0.01 to 20%, preferably from 0.2 to 10%, more preferably from 0.2 to 5%. According to a particular embodiment, the degree of substitution of the hyaluronic acid by the hydrophobic groups is within a range from 0.2 to 1%, preferably from 0.2 to 0.5%. According to another embodiment, the degree of substitution of the hyaluronic acid by the hydrophobic groups is within a range from 20 to 60%, preferably from 25 to 55%, more preferably from 30 to 50%. The degree of substitution reflects the number of hydrophobic groups bonded to 100 saccharide units of the hyaluronic acid chain. It is determined by the experimental conditions of the grafting and can be measured by nuclear magnetic resonance (NMR) or by elemental analysis for example. This parameter has an influence on the size of the particles.

According to one embodiment, the hyaluronic acid modified by hydrophobic groups is comprised of at least 3 disaccharide units, i.e. n is at least equal to 3, with the molar weight being in particular within a range from 100 Da to 1,000,000 kDa; preferably, the molar weight thereof is within a range from 100 Da to 3,000,000 Da; preferably from 5 k Da to 5,000 kDa; more preferably, is equal to 7.5 kDa, 51 kDa or 2,000 kDa. According to one embodiment, the hydrophobised hyaluronic acid is of low molar weight, i.e. having a molar weight less than or equal to 15 kDa. According to one embodiment, the hydrophobised hyaluronic acid is of a medium molar weight, i.e. having a molar weight ranging from 15 kDa to 100 kDa. According to one embodiment, the hydrophobised hyaluronic acid is of a high molar weight, i.e. having a molar weight greater than 100 kDa, more preferably greater than 1,000 kDa. According to a particular embodiment, the hydrophobised hyaluronic acid has a molar weight ranging from 100 kDa to 1,000 kDa, preferably from 100 kDa to 500 kDa. According to another particular embodiment, the hydrophobised hyaluronic acid has a molar weight ranging from 1,000 kDa to 5,000 kDa, preferably from 2,000 kDa to 4,000 kDa.

According to one embodiment, the particles of the invention comprise hydrophobised hyaluronic acid of the formula (I) having:
a high molar weight, ranging from 1,000 kDa to 5,000 kDa, preferably from 2,000 kDa to 4,000 kDa; and
a substitution rate ranging from 0.01 to 20%, preferably from 0.2 to 5%, more preferably from 0.2 to 1%, even more preferably ranging from 0.2 to 0.5%.

According to another embodiment, the particles of the invention comprise hydrophobised hyaluronic acid of the formula (I) having:
a medium molar weight, ranging from 15 kDa to 1,000 kDa, preferably from 30 kDa to 70 kDa; and
a substitution rate ranging from 0.01 to 20%, preferably from 0.1 to 5%, more preferably from 0.15 to 1.5%, even more preferably ranging from 0.15 to 1.0%.

According to another embodiment, the particles of the invention comprise hydrophobised hyaluronic acid of the formula (I) having:
a low molar weight, less than 15 kDa, preferably ranging from 1 to 10 kDa, more preferably ranging from 5 to 10 kDa; and
a substitution rate ranging from 0.01 to 20%, preferably from 0.1 to 5%, more preferably from 0.2 to 1.5%, even more preferably ranging from 0.2 to 1.0%.

According to another embodiment, the particles of the invention comprise hydrophobised hyaluronic acid of the formula (I) with controlled hydrophobicity. The hydrophobicity of the hydrophobised hyaluronic acid of the invention can be evaluated using infrared spectroscopy. In particular, the intensity of the characteristic infrared absorption bands of the alkyl functions is representative of the hydrophobicity. Intense absorption bands are synonymous with a substantial hydrophobicity which does not make it possible to obtain particles, while less intense bands are characteristic of hydrophobised hyaluronic acid that alone can form particles.

As such, according to one embodiment, the particles of the invention contain hydrophobised hyaluronic acid of the formula (I) having characteristic infrared spectroscopic absorption bands of the alkyl functions having a low or medium intensity.

The characteristic infrared spectroscopic absorption bands of the alkyl functions have wave numbers of about 2,800-3,000 cm⁻¹. The characteristic infrared spectroscopic absorption bands of the hydroxyl functions have wave numbers of about 3,200-3,700 cm⁻¹. The infrared spectra can be recorded on conventional devices known to those skilled in the art. In particular a Fourier transform infrared spectrometer can be used.

The relative intensity of the absorption bands of unsubstituted alkyls and hydroxyls of the hyaluronic acid can be representative of the hydrophobicity of the hydrophobised hyaluronic acid of the invention.

According to one embodiment, the particles of the invention contain hyaluronic acid modified by hydrophobic groups or the salts and/or derivatives thereof, having the formula (Ia):

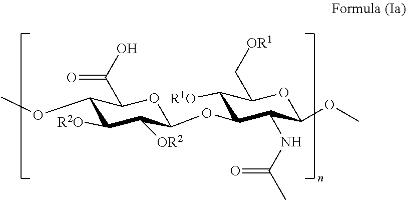

wherein n represents the number of disaccharide units of D-N-glucuronic acid and of N-acetyl glucosamine and represents a strictly positive integer;
R¹ and R² each independently represent a hydrogen atom, an R group or a —COR group wherein R represents:
   a linear or branched alkyl group, preferably linear, containing from 1 to 20 carbon atoms, preferably from 6 to 20 carbon atoms, more preferably of 7, 9, 11, 13, 15, 17 or 19 carbon atoms; or
   a linear or branched alkene group, preferably linear, containing 2 to 20 carbon atoms and bearing at least one C═C double bond; preferably containing 6 to 20 carbon atoms, more preferably 7, 9, 11, 13, 15, 17 or 19 carbon atoms;
with the condition that at least one of R¹ or R² represents a —COR group;
   said the hyaluronic acid modified by hydrophobic groups of the formula (I) having:
   a high molar weight, ranging from 1,000 kDa to 5,000 kDa, preferably from 2,000 kDa to 4,000 kDa; and
   a substitution rate ranging from 0.01 to 20%, preferably from 0.2 to 5%, more preferably from 0.2 to 1%, even more preferably ranging from 0.2 to 0.5%.

According to one embodiment, the particles of the invention comprise hyaluronic acid modified by hydrophobic groups or the salts and/or derivatives thereof, having the formula (Ia):

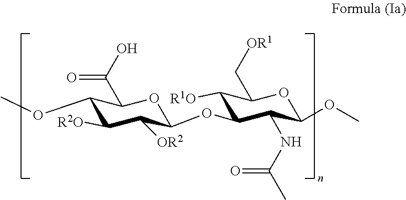

wherein n represents the number of disaccharide units of D-N-glucuronic acid and of N-acetyl glucosamine and represents a strictly positive integer;
R¹ and R² each independently represent a hydrogen atom, an R group or a —COR group wherein R represents:
   a linear or branched alkyl group, preferably linear, containing from 1 to 20 carbon atoms, preferably from 6 to 20 carbon atoms, more preferably of 7, 9, 11, 13, 15, 17 or 19 carbon atoms; or
   a linear or branched alkene group, preferably linear, containing 2 to 20 carbon atoms and bearing at least one C═C double bond; preferably containing 6 to 20 carbon atoms, more preferably 7, 9, 11, 13, 15, 17 or 19 carbon atoms;
with the condition that at least one of R¹ or R² represents a —COR group;
   said the hyaluronic acid modified by hydrophobic groups of the formula (I) having:
   a medium molar weight, ranging from 15 kDa to 1,000 kDa, preferably from 30 kDa to 70 kDa; and
   a substitution rate ranging from 0.01 to 20%, preferably from 0.1 to 5%, more preferably from 0.15 to 1.5%, even more preferably ranging from 0.15 to 1.0%.

According to an embodiment, the particles of the invention comprise hyaluronic acid modified by hydrophobic groups or the salts and/or derivatives thereof, having the formula (Ia):

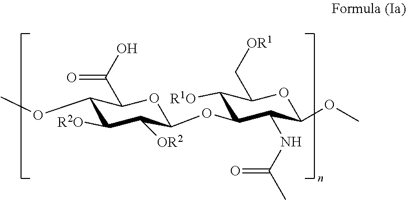

wherein n represents the number of disaccharide units of D-N-glucuronic acid and of N-acetyl glucosamine and represents a strictly positive integer;
R¹ and R² each independently represent a hydrogen atom, an R group or a —COR group wherein R represents:
   a linear or branched alkyl group, preferably linear, containing from 1 to 20 carbon atoms, preferably from 6 to 20 carbon atoms, more preferably of 7, 9, 11, 13, 15, 17 or 19 carbon atoms; or
   a linear or branched alkene group, preferably linear, containing 2 to 20 carbon atoms and bearing at least one C═C double bond; preferably containing 6 to 20 carbon atoms, more preferably 7, 9, 11, 13, 15, 17 or 19 carbon atoms;
with the condition that at least one of R¹ or R² represents a —COR group;
   said the hyaluronic acid modified by hydrophobic groups of the formula (I) having:
   a low molar weight, less than 15 kDa, preferably ranging from 1 to 10 kDa, more preferably ranging from 5 to 10 kDa; and
   a substitution rate ranging from 0.01 to 20%, preferably from 0.1 to 5%, more preferably from 0.2 to 1.5%, even more preferably ranging from 0.2 to 1.0%.

In one embodiment, the particles of the invention are microparticles or nanoparticles containing hyaluronic acid modified by hydrophobic groups of the formula (I).

According to one embodiment, the particles of the invention are microparticles or nanoparticles comprised of hyaluronic acid modified by hydrophobic groups of the formula (I).

According to one embodiment, the particles containing hydrophobised hyaluronic acid have a size between 1 nm to 100,000 nm; preferably, from 10 nm to 1,000 nm; preferably, from 1,000 nm to 100,000 nm; more preferably less than 500 nm.

According to one embodiment, the particles are constituted of at least two polysaccharides modified by hydrophobic groups of the formula (I). According to one embodiment, the particles are constituted of a mixture of at least two polysaccharides chosen from hyaluronic acids modified by hydrophobic groups of the formula (I).

According to one embodiment, the particles are particles of O-palmitoyl-hyaluronic acid, i.e. particles comprising hyaluronic acid modified by the palmitic acid by an O-acylation reaction.

According to one embodiment, the particles are particles of O-oleoyl-hyaluronic acid, i.e. particles comprising hyaluronic acid modified by the oleic acid by an O-acylation reaction.

According to one embodiment, the particles are particles of O-stearyl-hyaluronic acid, i.e. particles comprising hyaluronic acid modified by the stearic acid by an O-acylation reaction.

According to one embodiment, the particles are particles of O-lauryl-hyaluronic acid, i.e. particles comprising hyaluronic acid modified by the lauric acid by an O-acylation reaction.

According to one embodiment, the particles are particles of O-palmitoyl-hyaluronic acid, of O-oleoyl-hyaluronic acid, of O-stearyl-hyaluronic acid or of O-lauryl-hyaluronic acid.

According to one embodiment the particles comprising hydrophobised hyaluronic acid further comprise a cosmetic agent; in particular a depigmenting, healing, moisturising, perfuming, deodorising, anti-perspirant, cleaning, colouring, preserving, volumising and/or plumping, tensing agent.

Method of Preparing Particles

This invention also relates to a method of preparing particles of hydrophobised hyaluronic acid.

According to one embodiment, the method of preparing particles of hydrophobised hyaluronic acid comprises a step of mixing of at least:
two hydrophobised hyaluronic acids having the formula (I):

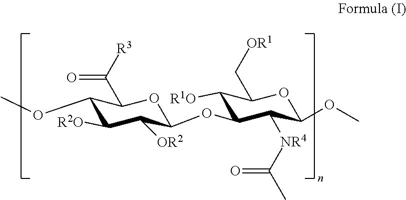

wherein R¹, R², R³, R⁴ and n are defined as hereinabove.

According to one embodiment, the method of forming particles of the invention comprises the dispersion of hydrophobised hyaluronic acid of the formula (I) according to the invention, in an aqueous dispersion medium.

According to one embodiment, the aqueous dispersion medium for the formation of particles is selected from: water, a buffer or the mixture thereof. In particular, the buffer can be a phosphate buffer.

According to a particular embodiment, the method of preparing particles is carried out under stirring, in particular magnetic, at a stirring speed between 50 to 1,000 rpm; preferably, at a stirring speed between 100 to 500 rpm; more preferably, at a stirring speed approximately equal to 200 rpm.

According to a particular embodiment, the method of preparing particles is carried out at a temperature between 4 to 100° C.; preferably, at a temperature between 10 to 35° C.; more preferably, at a temperature approximately equal to 20° C. According to a particularly preferred embodiment, the method of preparing particles of the invention is carried out at ambient temperature.

According to a particular embodiment, the method of preparing particles is carried out with a duration of stirring within a range from 2 hours to 15 days; preferably, this duration is within a range from 24 hours to 48 hours; more preferably, is approximately equal to 36 hours.

According to one embodiment, the hydrophobised hyaluronic acid of the formula (I) according to the invention is at a final concentration in the aqueous dispersion medium from 0.1 to 5% by weight, preferably from 0.25 to 2% by weight.

This invention also relates to particles of hydrophobised hyaluronic acid obtained by the method of preparing particles hereinabove.

Method of Preparing Hydrophobised Hyaluronic Acid of the Formula (I)

This invention also relates to a method of preparing hydrophobised hyaluronic acid of the formula (I) according to the invention, wherein at least one of R¹ or R² represents a —COR group. According to one embodiment, the method of the invention comprises a step of O-acylation between the hyaluronic acid and a fatty acid or a fatty acid derivative.

According to a preferred embodiment, the step of O-acylation is carried out at ambient temperature. Controlling the temperature of O-acylation advantageously makes it possible to modulate the final degree of substitution of the hydrophobised hyaluronic acid of the invention and as such control the hydrophobicity thereof.

According to a particular embodiment, the step of O-acylation is carried out under stirring, in particular magnetic, at a stirring speed between 50 to 1,000 rpm; preferably, at a stirring speed between 100 to 500 rpm; more preferably, at a stirring speed approximately equal to 250 rpm.

According to a particular embodiment, the step of O-acylation is carried out for a duration ranging between 2 hours to 5 days; preferably, this duration is within a range from 48 hours to 72 hours; more preferably, is approximately equal to 72 hours.

Composition Comprising Particles of Hyaluronic Acid

This invention also relates to compositions comprising particles of hydrophobised hyaluronic acid of the formula (I) such as defined hereinabove, in association with a pharmaceutically or cosmetically acceptable excipient. Preferably, the particles entering into the compositions of the invention are particles which are entirely or substantially comprised of hydrophobised hyaluronic acid of the formula (I) such as defined hereinabove. According to a preferred embodiment, the particles entering into the compositions of the invention do not comprise cyclodextrin.

According to one embodiment, the compositions of the invention include a quantity of said particles within a range from 10⁻¹⁰% to 100% by weight with respect to the total weight of the composition, preferably from 0.0001% to 50%, very preferably from 0.001% and 10%, by weight with respect to the total weight of the composition.

According to one embodiment, the composition is in solid form, or in the form of a solution, suspension or emulsions such as water in oil (W/O), oil in water (O/W) or water in oil in water (W/O/W) emulsions, wherein the oil is of a plant or mineral nature, in a biocompatible medium.

According to one embodiment, the composition further comprises a solvent; more preferably biocompatible. According to an embodiment, the composition further comprises a solvent chosen from aqueous or hydro-alcoholic solutions.

According to one embodiment, the composition further comprises at least one cosmetically acceptable excipient, that can be used in the form of gels, pastes, salves, lotions, creams, milks, sticks, shampoos, powders, aerosols, patches.

Generally, the compositions implemented in the method of cosmetic treatment of the human body further comprise many types of additives or active ingredients chosen from at least one of the following ingredients: greasy substance, organic solvents, thickening agents, gelling agents, softening agents, detergent surfactants, superfatting agents, thickening and/or gelling surfactants, antioxidants, opacifiers, stabilisers, foaming agents, perfumes, emulsifying surfactants, hydrotropic agents, plasticisers, superfatting agents, texturing agents, pigments, sequestrants, chelating agents, preservatives, chemical filters or mineral filters, essential oils, colorants, pigments, lipophilic and hydrophilic actives, humectants, for example glycerin, preservatives, colouring agents, perfumes, cosmetic actives, mineral or organic solar filters, mineral fillers such as iron oxides, titanium oxides and talc, synthetic fillers such as nylons and poly(methyl methacrylate) crosslinked or not, silicone elastomers, sericites or plant extracts or lipid vesicles or any other ingredient usually used in cosmetics.

According to one embodiment, the compositions containing hydrophobised hyaluronic acid of the formula (I) are administered directly topically.

According to one embodiment, the compositions containing hydrophobised hyaluronic acid of the formula (I) are administered indirectly topically. The term “indirectly topically” means the use of a fabric support or of non-woven materials to administer the composition.

Uses

The invention has for object the use of particles comprising hyaluronic acid modified by hydrophobic groups, or the salts and/or derivatives thereof. In particular, the use of particles comprising hyaluronic acid modified by hydrophobic groups or the salts and/or derivatives thereof, having the formula (I):

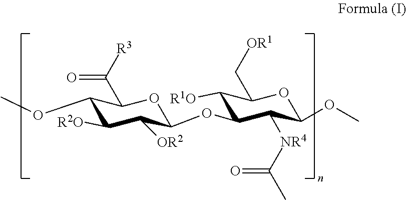

wherein
R¹ and R² each independently represent a hydrogen atom, an R group or a —COR group wherein R represents:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 carbon atoms; or
   a linear or branched alkene group, containing 2 to 1,000 carbon atoms and bearing at least one C═C double bond;
R³ represents:
   a hydroxyl group;
   a —O⁻X⁺ group wherein X⁺ represents a cation; preferably, a sodium ion; or
   a —OR′ group or a —NHR′ group wherein R′ represents:
      a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
      a linear or branched alkene group, containing 2 to 1,000 carbon atoms and bearing at least one C═C double bond;
R⁴ represents a hydrogen atom or an R″ group chosen from:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 carbon atoms; or
   a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group.

This invention also relates to the use of particles constituted entirely or substantially of hyaluronic acid modified by hydrophobic groups of the formula (I); preferably, linear or branched alkyl or alkene groups, in particular linear containing from 1 to 60 carbon atoms; more preferably, containing from 1 to 20 carbon atoms.

This invention relates to the use of hyaluronic acid modified by hydrophobic groups; preferably, linear or branched alkyl or alkene groups, in particular linear containing from 1 to 60 carbon atoms; more preferably, containing from 1 to 20 carbon atoms.

In particular, this invention relates to the use of particles comprising hydrophobised hyaluronic acid, or the salts and/or derivatives thereof, obtained by a method of preparation that comprises a step of mixing of at least:
two hydrophobised hyaluronic acids having the formula (I):

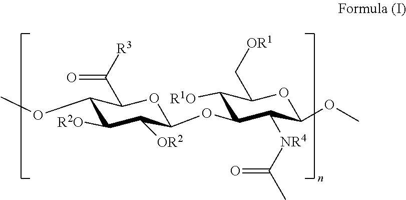

wherein R¹, R², R³, R⁴ and n are defined as hereinabove.

The invention also has for object the use of particles comprising hyaluronic acid modified by hydrophobic groups, or the salts and/or derivatives thereof, the field of cosmetics and/or pharmaceuticals, in particular the dermatological field. According to one embodiment, the particles used in the cosmetic field and/or the pharmaceutical field are particles comprised of hyaluronic acid. According to one embodiment, the particles used in the cosmetic field and/or the pharmaceutical field are particles that do not comprise cyclodextrin.

The details of the products, of the compositions and of the methods described hereinabove apply to the cosmetic and/or pharmaceutical uses, in particular dermatological described hereinbelow.

According to a first aspect, the invention relates to the use in the cosmetics field of particles of hyaluronic acid modified by hydrophobic groups, or the salts and/or derivatives thereof, having the formula (I):

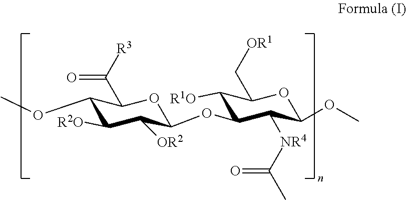

wherein
R¹ and R² each independently represent a hydrogen atom, an R group or a —COR group wherein R represents:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
R³ represents:
   a hydroxyl group;
   a —O⁻X⁺ group wherein X⁺ represents a cation; preferably, a sodium ion; or
   a —OR′ group or a —NHR′ group wherein R′ represents:
      a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
      a linear or branched alkene group, containing 2 to 1,000 carbon atoms and bearing at least one C═C double bond;
R⁴ represents a hydrogen atom or an R″ group chosen from:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group.

According to one embodiment, the invention has for object the cosmetic use of particles comprising hyaluronic acid modified by hydrophobic groups of the formula (I) as a cosmetic agent; preferably, as a volumising agent and/or plumping agent. According to an embodiment, the particles of O-palmitoyl-hyaluronic acid, of O-oleoyl-hyaluronic acid, of O-stearyl-hyaluronic acid or of O-lauryl-hyaluronic acid are useful as a cosmetic agent; preferably, as a volumising agent and/or plumping agent. According to an embodiment, the particles of O-palmitoyl-hyaluronic acid are useful as a cosmetic agent; preferably, as a volumising agent and/or plumping agent.

In the framework of the revealing of the “volumising” effect and/or of the “plumping” effect of the particles of hydrophobised hyaluronic acid of the formula (I), the Applicant has revealed the action of the particles of the invention for the action thereof in the epidermis and the dermis. In particular, the Applicant has revealed the action of the particles of the invention as a stimulating agent for the endogenous production of hyaluronic acid. The particles of the invention as such advantageously make it possible to combat the thinning of the skin.

According to one embodiment, the invention has for object the cosmetic use of particles of hydrophobised hyaluronic acid of the formula (I) characterised in that the particles of hydrophobised hyaluronic acid of the formula (I) furthermore favour the expansion of the connecting tissues of the human skin; preferably, of the epidermis and of the dermis. In this invention, the term “expansion of the connecting tissues of the human skin” means any increase in the weight and/or in the volume and/or in the thickness of the connecting tissues of the human skin; preferably, of the epidermis and of the dermis. According to one embodiment, the particles of O-palmitoyl-hyaluronic acid, of O-oleoyl-hyaluronic acid, of O-stearyl-hyaluronic acid or of O-lauryl-hyaluronic acid favour the expansion of the connecting tissues of the human skin; preferably, of the epidermis and of the dermis. According to one embodiment, the particles of O-palmitoyl-hyaluronic acid favour the expansion of the connecting tissues of the human skin; preferably, of the epidermis and of the dermis.

According to one embodiment, the invention has for object the cosmetic use of particles of hydrophobised hyaluronic acid for limiting and/or correcting imperfections of the skin; preferably, as a tensing agent of the skin or as an agent that favours the filling in of wrinkles and/or lines of the skin. According to one embodiment, the particles of O-palmitoyl-hyaluronic acid, of O-oleoyl-hyaluronic acid, of O-stearyl-hyaluronic acid or of O-lauryl-hyaluronic acid are useful for limiting and/or correcting imperfections of the skin; preferably, as a tensing agent of the skin or as an agent that favours the filling in of wrinkles and/or lines of the skin. According to one embodiment, the particles of O-palmitoyl-hyaluronic acid are useful for limiting and/or correcting imperfections of the skin; preferably, as a tensing agent of the skin or as an agent that favours the filling in of wrinkles and/or lines of the skin.

According to one embodiment, the invention has for object the cosmetic use of particles of hydrophobised hyaluronic acid of the formula (I), characterised in that the particles of hydrophobised hyaluronic acid of the formula (I) furthermore stimulate an increase in the elasticity of the skin. According to one embodiment, the particles of O-palmitoyl-hyaluronic acid, of O-oleoyl-hyaluronic acid, of O-stearyl-hyaluronic acid or of O-lauryl-hyaluronic acid are useful for increasing the elasticity of the skin. According to one embodiment, the particles of O-palmitoyl-hyaluronic acid are useful for increasing the elasticity of the skin.

According to one embodiment, the invention has for object the cosmetic use of particles of hydrophobised hyaluronic acid in order to improve the moisturising of the mucous members, of the epidermis and of the dermis, in particular for improving the moisturising of the epidermis and of the dermis. According to one embodiment, the particles of hydrophobised hyaluronic acid are useful for countering the drying out of the mucous membranes. According to one embodiment, the particles of hydrophobised hyaluronic acid are useful for countering the drying out of the skin; preferably, of the epidermis and of the dermis. According to one embodiment, the particles of O-palmitoyl-hyaluronic acid, of O-oleoyl-hyaluronic acid, of O-stearyl-hyaluronic acid or of O-lauryl-hyaluronic acid are useful for improving the moisturising of the epidermis and of the dermis. According to one embodiment, the particles of O-palmitoyl-hyaluronic acid are useful for improving the moisturising of the epidermis and of the dermis. According to one embodiment, the particles of O-palmitoyl-hyaluronic acid, of O-oleoyl-hyaluronic acid, of O-stearyl-hyaluronic acid or of O-lauryl-hyaluronic acid are useful for countering the drying out of the skin; preferably, of the epidermis and of the dermis. According to one embodiment, the particles of O-palmitoyl-hyaluronic acid are useful for countering the drying out of the skin; preferably, of the epidermis and of the dermis.

According to one embodiment, the uses of the particles of this invention are intended for mature and/or aged skin.

According to one embodiment, the invention has for object the cosmetic use of particles containing hydrophobised hyaluronic acid as encapsulating agents of cosmetically active ingredients. According to one embodiment, the invention has for object the cosmetic use of particles containing hydrophobised hyaluronic acid as inclusion complexes of cosmetically active ingredients.

According to one embodiment, the particles of hydrophobised hyaluronic acid are not used as encapsulating agents. According to one embodiment, the particles of hydrophobised hyaluronic acid are not used as inclusion complexes.

According to one embodiment, the invention relates to a method of cosmetic treatment of the human body. In particular, the invention related to the use of particles containing hydrophobised hyaluronic acid, or the salts and/or derivatives thereof, as a cosmetic agent.

According to a second aspect, the invention relates to particles containing hyaluronic acid modified by hydrophobic groups or the salts and/or derivatives thereof, for the use thereof in the pharmaceutical field, as a medicament, having the formula (I):

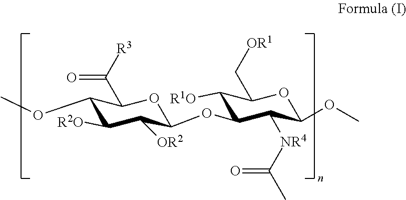

wherein
R¹ and R² each independently represent a hydrogen atom, an R group or a —COR group wherein R represents:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
R³ represents:
   a hydroxyl group;
   a —O⁻X⁺ group wherein X⁺ represents a cation; preferably, a sodium ion; or
   a —OR′ group or a —NHR′ group wherein R′ represents:
      a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
      a linear or branched alkene group, containing 2 to 1,000 carbon atoms and bearing at least one C═C double bond;
R⁴ represents a hydrogen atom or an R″ group chosen from:
   a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
   a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group.

According to one embodiment, the invention has for object particles of hyaluronic acid modified by hydrophobic groups of the formula (I) for the use thereof as a dermatological agent.

According to one embodiment, the invention has for object particles of hyaluronic acid modified by hydrophobic groups of the formula (I) for the use thereof as a dermatological agent; preferably, as a healing agent; more preferably as a healing agent of superficial wounds, in particular of the skin or of the mucous membranes. According to one embodiment, the particles of O-palmitoyl-hyaluronic acid, of O-oleoyl-hyaluronic acid, of O-stearyl-hyaluronic acid or of O-lauryl-hyaluronic acid are useful as a dermatological agent; preferably, as a healing agent of superficial wounds. According to one embodiment, the particles of O-palmitoyl-hyaluronic acid are useful as a dermatological agent; preferably, as a healing agent of superficial wounds.

According to one embodiment, the invention has for object particles containing hydrophobised hyaluronic acid for the use thereof as a dermatological agent; preferably, for limiting and/or correcting imperfections of the skin. According to one embodiment, the particles of O-palmitoyl-hyaluronic acid, of O-oleoyl-hyaluronic acid, of O-stearyl-hyaluronic acid or of O-lauryl-hyaluronic acid are useful as a dermatological agent; preferably, for limiting and/or correcting imperfections of the skin. According to one embodiment, the particles of O-palmitoyl-hyaluronic acid are useful as a dermatological agent; preferably, for limiting and/or correcting imperfections of the skin.

According to one embodiment, the invention has for object particles of hydrophobised hyaluronic acid of the formula (I) for the use thereof as a dermatological agent, characterised in that the particles of hydrophobised hyaluronic acid of the formula (I) furthermore stimulate an increase in the elasticity of the skin. According to one embodiment, the particles of O-palmitoyl-hyaluronic acid, of O-oleoyl-hyaluronic acid, of O-stearyl-hyaluronic acid or of O-lauryl-hyaluronic acid are useful for increasing the elasticity of the skin. According to one embodiment, the particles of O-palmitoyl-hyaluronic acid are useful for increasing the elasticity of the skin.

According to one embodiment, the invention has for object particles of hydrophobised hyaluronic acid for the use thereof as a dermatological agent, characterised in that the particles of hydrophobised hyaluronic acid of the formula (I) improve the moisturising of the mucous membranes, epidermis and dermis, in particular of the epidermis and of the dermis. According to one embodiment, the particles of hydrophobised hyaluronic acid are useful for countering the drying out of the mucous membranes. According to one embodiment, the particles of hydrophobised hyaluronic acid are useful for countering the drying out of the skin; preferably, of the epidermis and of the dermis. According to one embodiment the particles of O-palmitoyl-hyaluronic acid, of O-oleoyl-hyaluronic acid, of O-stearyl-hyaluronic acid or of O-lauryl-hyaluronic acid are useful for improving the moisturising of the epidermis and of the dermis. According to one embodiment, the particles of O-palmitoyl-hyaluronic acid are useful for improving the moisturising of the epidermis and of the dermis. According to one embodiment, the particles of O-palmitoyl-hyaluronic acid, of O-oleoyl-hyaluronic acid, of O-stearyl-hyaluronic acid or of O-lauryl-hyaluronic acid are useful for countering the drying out of the skin; preferably, of the epidermis and of the dermis. According to one embodiment, the particles of O-palmitoyl-hyaluronic acid are useful for countering the drying out of the skin; preferably, of the epidermis and of the dermis.

According to one embodiment, the invention has for object particles of hydrophobised hyaluronic acid for the use thereof as a dermatological agent; preferably, as encapsulating agents. According to one embodiment, the invention has for object particles of hydrophobised hyaluronic acid for the use thereof as a dermatological agent; preferably, as inclusion complexes.

According to one embodiment, the particles of hydrophobised hyaluronic acid are not used as encapsulating agents of dermatologically active ingredients. According to one embodiment, the particles of hydrophobised hyaluronic acid are not used as inclusion complexes of dermatologically active ingredients.

According to one embodiment, the invention relates to particles containing hyaluronic acid modified by hydrophobic groups, or the salts and/or derivatives thereof, in the preparing of compositions for the dermatological treatment of an individual; more preferably for the treatment of the epidermis and/or of the dermis.

### BRIEF DESCRIPTION OF THE FIGURES

FIG. 1 shows the IR spectrum characteristic of native hyaluronic acid (FIG. 1A) and of the hyaluronic acid modified by hydrophobic groups according to the example 1.1.1 (FIG. 1B), 1.2 (FIG. 1C), 1.3 (FIG. 1D), or 1.4 (FIG. 1E).

FIG. 2 is a graph showing the intensity of penetration in the epidermis (EP) and the papillary dermis (DP) of the hydrophobised hyaluronic acid for the formulation A1 tested in the example 3.1.

### EXAMPLES

This invention will be understood better when reading the following examples which show the invention in a non-limiting manner.

Abbreviations

° C.: degrees Celsius

Dₕ: hydrodynamic diameter

DMF: dimethylformamide

HABP: anti-hyaluronic acid binding protein

IR: infrared

mL: millilitre

rpm: revolutions per minute

A. Chemical Part

Reagents

All of the chemical reagents and solvents were obtained from commercial sources

(Aldrich, Acros, Life Core) and were used as is. The spectroscopic quality solvents were purchased from the supplier Aldrich.

Infrared Spectroscopy (ATR-FTIR)

The spectra were recorded as a function of time under a flow of nitrogen using an JASCO FT/IR-4100 infrared spectrometer. The principle consists in placing a crystal (diamond) in contact with the sample to be analysed before it is passed through by an infrared beam.

Measurements of the Size of the Particles

The sizes of the particles were evaluated by the hydrodynamic diameter. The measurements of the average hydrodynamic diameters of the nanoparticles and microparticles were taken with a Zetasizer nanoseries Nano-ZS90 from Malvern instruments SA (Orsay, France) via quasi-elastic diffusion of light.

### Example 1

### Synthesis and Characterisation of Hydrophobised Hyaluronic Acids

1.1. Synthesis of O-Palmitoyl Hyaluronic Acid

1.1.1. Synthesis of O-Palmitoyl Hyaluronic Acid 2,000 kDa (AH-4-AP)

2 g (1 weight equivalent) of sodium hyaluronate (molar weight of approximately 2,000 kDa) are treated with 40 mL of DMF then palmitic acid chloride is added to the reaction medium (3 weight equivalents). A white suspension is obtained. The mixture is stirred (under magnetic stirring at a speed of 250 rpm) for 3 days at 20° C. 200 mL of methanol is added to the mixture. The powder is then isolated by filtration on sintered glass, washed with methanol and with acetone, then is dried in a vacuum at ambient temperature for one night. A white powder is obtained.

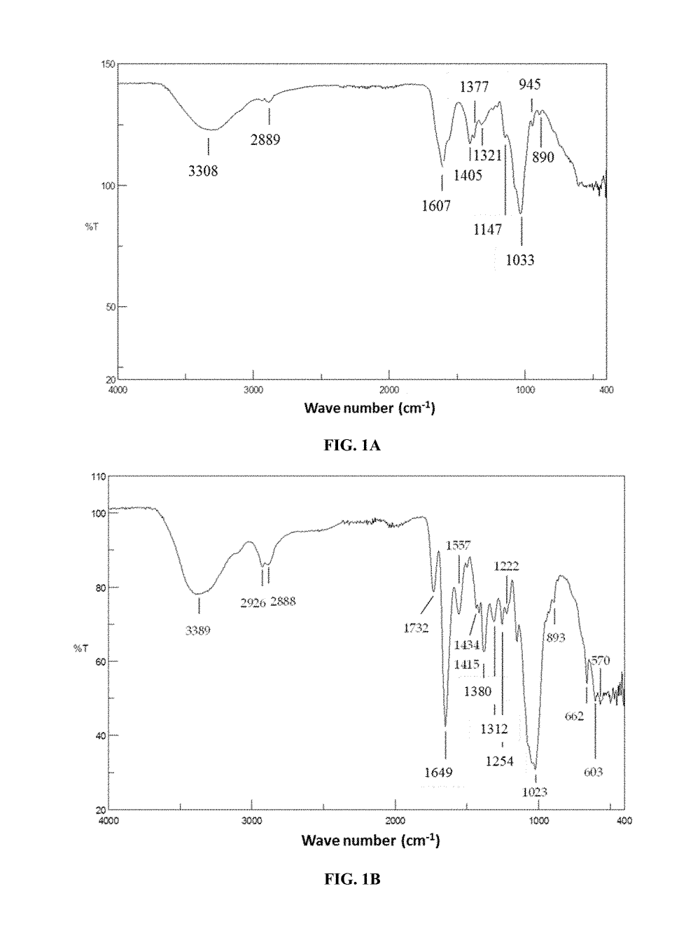

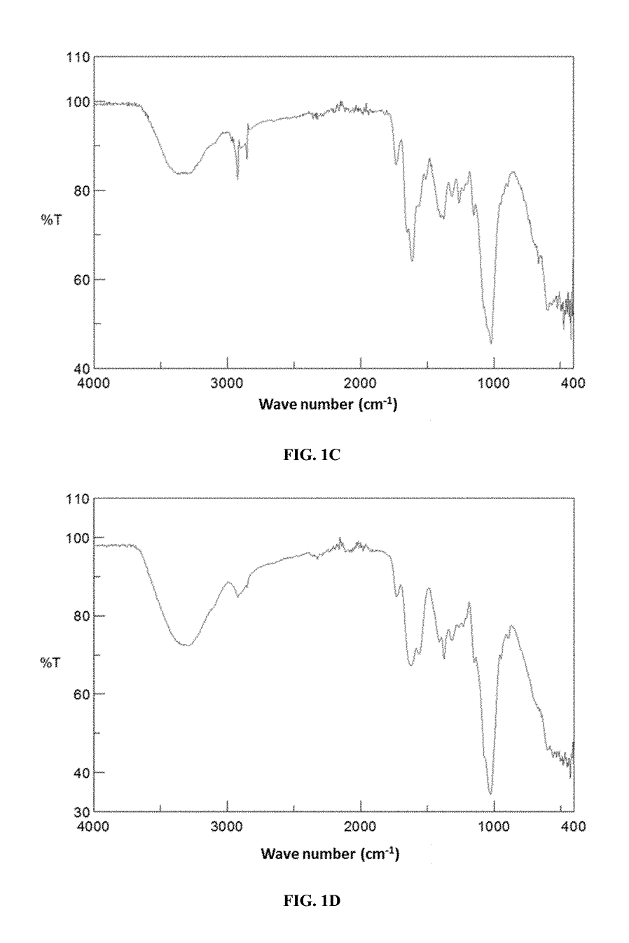

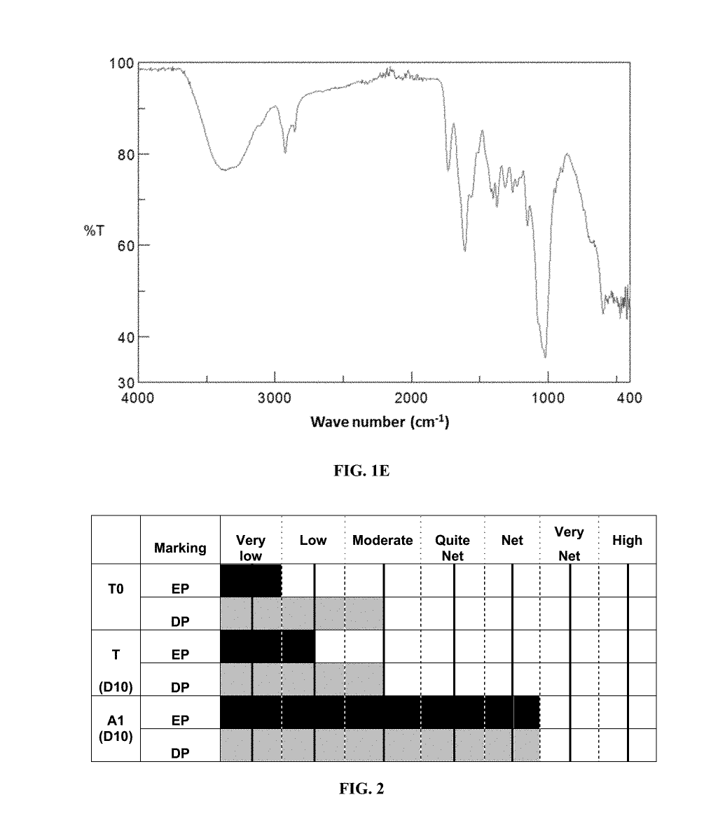

The product was characterised by infrared spectrometry (IR). The analysis via IR spectroscopy is a fast and effective method for identifying the chemical groups of the hydrophobised hyaluronic acid (FIG. 1B) by comparison with the spectrum of the native hyaluronic acid (FIG. 1A).

This analysis made it possible to reveal the presence of bands corresponding to the carbonyl groups of the ester function. The bands at 1023 cm⁻¹ and 1254 cm⁻¹ correspond to the C—O groups of the ester function. The spectrum of the O-palmitoyl hyaluronic acid also showed bands at 2,888 cm⁻¹ and 2,926 cm⁻¹ corresponding to the alkyl groups of the palmitic acid.

The product was also characterised by elemental analysis using an LECO SC144 analyser. 20-mg samples were burnt at 1,350° C. under a flow of oxygen. This technique makes it possible to determine the number of carbon, hydrogen and oxygen atoms present in the molecule and as such recover the degree of substitution (DS) of the hyaluronic acid by the hydrophobic chains, according to the following calculation:

$$DS = {( \frac{C \% mol}{O \% mol} )}_{Hydrophobised HA} - {( \frac{C \% mol}{O \% mol} )}_{Native HA}$$

| Product | C % | H % | O % | C/O (% mol) | DS (%) |
|---|---|---|---|---|---|
| native AH | 36.05 | 5.53 | 46.24 | 1.04 | 0 |
| 2000 | | | | | |
| AH-4-AP | 43.15 | 6.08 | 41.23 | 1.40 | 0.36 |

1.1.2. Synthesis of O-Palmitoyl Hyaluronic Acid 51 kDa

1 g of sodium hyaluronate (molar weight of approximately 51 kDa) are treated with 20mL of dimethylformamide then 0.684 g of palmitic acid chloride (1 eq.). A white suspension is obtained. The mixture is stirred (magnetic 250 rpm) for 3 days at 20° C. The aspect of the suspension remains unchanged. After the 3 days of reaction, 50 mL of methanol are added to the mixture. The powder is isolated by filtration (sintered glass with a porosity of 3), washed with methanol then with acetone and dried in a vacuum at ambient temperature for one night. A white powder is obtained that will be frozen then lyophilised for 24 h.

The product was characterised by elemental analysis and the degree of substitution calculated as in the example 1.1.1:

| Product | C % | H % | O % | C/O (% mol) | DS (%) |
|---|---|---|---|---|---|
| native AH 51 | 36.63 | 5.61 | 46.57 | 1.05 | 0 |
| AH(51)-4-AP | 42.43 | 6.13 | 43.86 | 1.29 | 0.24 |

1.1.3. Synthesis of O-Palmitoyl Hyaluronic Acid 7.5 kDa

1 g of sodium hyaluronate (molar weight of approximately 7.5 kDa) are treated with 20mL of dimethylformamide then 0.684 g of palmitic acid chloride (1 eq.). A white suspension is obtained. The mixture is stirred (magnetic 250 rpm) for 3 days at 20° C. The aspect of the suspension remains unchanged. After the 3 days of reaction, 50 mL of methanol are added to the mixture. The powder is isolated by filtration (sintered glass with a porosity of 4), washed with methanol then with acetone and dried in a vacuum at ambient temperature for one night. A white powder is obtained that will be frozen then lyophilised for 24 h.

The product was characterised by elemental analysis and the degree of substitution calculated as in the example 1.1.1:

| Product | C % | H % | O % | C/O (% mol) | DS (%) |
|---|---|---|---|---|---|
| Native AH 7,5 | 38.49 | 5.67 | 46.85 | 1.10 | 0 |
| AH(7,5)-4-AP | 43.44 | 6.56 | 37.49 | 1.54 | 0.45 |

1.2. Synthesis of O-Oleoyl Hyaluronic Acid (AH-4-AO)

2 g of sodium hyaluronate (molar weight of approximately 2,000 kDa) are treated with 40 mL of DMF then 4.106 g (3 eq.) oleic acid chloride (yellowish solution). A white suspension is obtained. The mixture is stirred (under magnetic stirring at a speed of 1,000 rpm) for 3 days at 26° C. 200 mL of methanol is added to the mixture. The powder is then isolated by filtration on sintered glass, washed with methanol and with acetone, then is dried in a vacuum at ambient temperature for one night. A white powder is obtained.

The product was characterised by infrared spectrometry (FIG. 1C). This analysis made it possible to reveal the presence of bands corresponding to the carbonyl groups of the ester function C═O at 1733 cm⁻¹. The spectrum of the O-oleoyl-hyaluronic acid also showed bands at 2852 and 2922 cm⁻¹ corresponding to the alkyl groups of the oleic acid.

1.3. Synthesis of O-Stearyl Hyaluronic Acid (AH-4-AS)

2 g of sodium hyaluronate (molar weight of approximately 2,000 kDa) are treated with 40 mL of DMF then 4.106 g (3 eq.) stearic acid chloride. A brown suspension is obtained. The mixture is stirred (under magnetic stirring at a speed of 1,000 rpm) for 3 days at 26° C. 200 mL of methanol is added to the mixture. The powder is then isolated by filtration on sintered glass, washed with methanol and with acetone, then is dried in a vacuum at ambient temperature for one night. A white powder is obtained.

The product was characterised by infrared spectrometry (FIG. 1D). This analysis made it possible to reveal the presence of bands corresponding to the carbonyl groups of the ester function C═O at 1731 cm⁻¹. The spectrum of the O-stearyl-hyaluronic acid also showed bands at 2850 and 2919 cm⁻¹ corresponding to the alkyl groups of the stearic acid.

1.4. Synthesis of O-Lauryl Hyaluronic Acid (AH-4-AL)

2 g of sodium hyaluronate (molar weight of approximately 2,000 kDa) are treated with 40 mL of DMF then 4.106 g (3 eq.) lauric acid chloride. A white suspension is obtained. The mixture is stirred (under magnetic stirring at a speed of 1,000 rpm) for 3 days at 26° C. 200 mL of methanol is added to the mixture. The powder is then isolated by filtration on sintered glass, washed with methanol and with acetone, then is dried in a vacuum at ambient temperature for one night. A white powder is obtained.

The product was characterised by infrared spectrometry (FIG. 1E). This analysis made it possible to reveal the presence of bands corresponding to the carbonyl groups of the ester function C═O at 1732 cm⁻¹. The spectrum of the O-lauryl-hyaluronic acid also showed bands at 2850 and 2924 cm⁻¹ corresponding to the alkyl groups of the lauric acid.

### Example 2

### Formation of Particles of Hydrophobised Hyaluronic Acid

2.1. Particles of O-Palmitoyl Hyaluronic Acid (Particles of AH-4-AP)

The O-palmitoyl-hyaluronic acid AH-4-AP obtained according to the example 1.1.1 is dispersed in water. The milieu is mixed for 72 h at ambient temperature in order to form the particles.

The results of the measurement of the size of the particles, expressed as an average size Z (nm) or as a percentage of the volume occupied by the particles are indicated in table 1:

| Parameter | Meas- | Meas- | Measurement | | Standard |
|---|---|---|---|---|---|
| followed | urement 1 | urement 2 | 3 | Means | deviation |
| Average | 435 | 365 | 295 | 365 | 70 |
| size Z | | | | | |
| (nm) | | | | | |
| % volume | 438 | 369 | 297 | 368 | 71 |

2.2. Particles of AH-4-AP, AH-4-AO, AH-4-AS and AH-4-AL

Particles were formed by dispersing chains of hydrophobised hyaluronic acid 2,000 kDa, such as described in the examples 1.1.1, 1.2, 1.3 and 1.4, in water.

The experimental conditions are specified in table 2. A dilution by 2 was carried out then the medium was stirred for 72 h at ambient temperature.

| Hydrophobised | Quantity of hydrophobised | Quantity of water in the |
|---|---|---|
| hyaluronic acid | hyaluronic acid (mg) | mixture (g) |
| AH-4-AL | 11.2 | 1.0018 |
| AH-4-AP | 10.1 | 1.0218 |
| AH-4-AS | 10.8 | 1.0061 |
| AH-4-AO | 10.5 | 1.0128 |

2.3. Particles of O-Palmitoyl Hyaluronic Acid 51 kDa or 7.5 kDa

Particles were formed by dispersing chains of 0-palmitoyl hyaluronic acid 51 kDa or 7.5 kDa, such as described in the examples 1.1.2 and 1.1.3, in water, at a concentration of 1% by weight.

B. Biology Part

### Example 3

### Measurement of the Penetration into the Epidermis and the Dermis of Particles of Hydrophobised Hyaluronic Acid

Moisturising and attenuating the effects of ageing of the skin (in particular, decreasing wrinkles and/or lines) requires having formulations available that comprise active compounds that can effectively penetrate the epidermis (EP) then the papillary dermis (DP) of an individual.

These experiments therefore have the purpose of showing that formulations that comprise particles of hydrophobised hyaluronic acid according to the invention, are able to effectively penetrate the epidermis and the dermis of an individual.

3.1. Particles of O-Palmitoyl-Hyaluronic Acid AH-4-AP

A formulation was studied for ten days on explants of human skin ex vivo:
Formulation A1: particles comprising O-palmitoyl-hyaluronic acid AH-4-AP (Molar weight of about 2 MDa) obtained according to the protocol of example 2.

The formulation A1 was stored at ambient temperature before and for the duration of the study.

9 explants with a diameter of approximately 11 mm (±1 mm) were prepared using an abdominoplasty from a woman aged 57 years of the Caucasian type (reference P1128-AB57). The explants were maintained in a BEM medium (BIO-EC's Explants Medium) at 37° C. in a wet atmosphere, enriched with 5% CO₂.

The formulation A1 was applied topically for 5 mg of product (spread with a small spatula) per explant, at days D0, D3, D5 and D7.

The explants of lots T0 and T did not receive any treatment other than the renewing of the medium.

The maintaining medium was renewed for half (1 ml) at D3, D5 and D7.

At D0, the explants of the lot T0 were sampled and separated into two. A half-explant was fixed in buffered formol and the other was frozen at −80° C.

At D10, the 3 explants of each lot were sampled and treated in the same way.

Table 3 shows the various lots tested.

| Lot | Treatment | Number of explants tested | Treatment stopped |
|---|---|---|---|
| T0 | None | 3 | D0 |
| T | None | 3 | D10 |
| A1 | formulation A1 | 3 | D10 |

The marking of the hyaluronic acid was carried out on Formol paraffin sections with an anti-hyaluronic acid binding protein (HABP) (Seikagaku, ref 400763-1A), at 1/100 for 1 h at ambient temperature with a biotin/streptavidin amplifier system, revealed by the commercial revelator Vector®VIP (SK 4600). The marking was carried out using an automatic immunolabelling machine (Dako, AutostainerPlus).

The marking was then evaluated via microscopic examination and quantified through image analysis using the CellD software.

The results shown in FIG. 2 show that:
the control lots at D0 and at D10 (respectively T0 and T) have a low marking of the hyaluronic acid in the epidermis and a medium marking in the papillary dermis;
the treatment of the explants with the formulation A1 containing hydrophobised hyaluronic acid AH-4-AP have a clear marking of the hyaluronic acid both in the epidermis and the papillary dermis.

In conclusion, these experiments show that the formulations containing hydrophobised hyaluronic acid, in particular O-palmitoyl-hyaluronic acid AH-4-AP, allow for good penetration of the hyaluronic acid both in the epidermis and in the dermis, even for high molecular weights of hyaluronic acid.

3.2. Particles of AH-4-AS or of AH-4-AO

Two other formulations were studied for nine days on explants of human skin ex vivo:
Formulation A2: particles comprising O-stearyl-hyaluronic acid (AH-4-AS) obtained according to the protocol of example 2,
Formulation A3: particles comprising O-oleoyl-hyaluronic acid (AH-4-AO) obtained according to the protocol of example 2.

The products were stored at ambient temperature before and for the duration of the study.

12 explants with a diameter of approximately 11 mm (±1 mm) were prepared using an abdominoplasty from a woman aged 70 years of the Caucasian type (reference P1474-AB70). The explants were maintained in a BEM medium (BIO-EC's Explants Medium) at 37° C. in a wet atmosphere, enriched with 5% CO₂.

The products were applied topically for 5 mg/cm² of product (spread with a small spatula) per explant, at days D0, D1, D4, D6 and D8.

The explants of lots T0 and T did not receive any treatment other than the renewing of the medium.

At D0, the explants of the lot T0 were sampled and separated into two. A half-explant was fixed in buffered formol and the other was frozen at −80° C.

At D9, the 3 explants of each lot were sampled and treated in the same way.

The tableau 4 shows the various lots tested.

| Lot | Treatment | Number of explants tested | Treatment stopped |
|---|---|---|---|
| T0 | None | 3 | D0 |
| T | None | 3 | D9 |
| A2 | formulation A2 | 3 | D9 |
| A3 | formulation A3 | 3 | D9 |

The marking of the hyaluronic acid was carried out on Formol paraffin sections with an anti-hyaluronic acid binding protein (HABP) (Seikagaku, ref 400763-1A), at 1/100 for 1 h at ambient temperature with a biotin/streptavidin amplifier system, revealed by the commercial revelator Vector®VIP (SK 4600). The marking was carried out using an automatic immunolabelling machine (Dako, AutostainerPlus).

The marking was then evaluated via microscopic examination and quantified through image analysis using the CellD software.

The results showed for formulations A2 and A3, a generally improved morphology in relation to the control lot after nine days. The epidermal layer is thicker and the relief of the dermo-epidermal junction is improved. In addition, the marking results show that the application of formulations A2 and A3 topically induce an improvement in the expression of the hyaluronic acid both in the epidermis and the dermis.

Consequently, the particles of hydrophobised hyaluronic acid of the invention have an anti-ageing activity.

### Example 4

### Study via the Analysis of Images of the Variation in Density and/or in Thickness of the Epidermis and/or of the Papillary Dermis in the Presence of Particles of Hydrophobised Hyaluronic Aacid

4.1 Particles of O-Palmitoyl-Hyaluronic acid AH-4-AP

The lots described hereinabove in the example 3.1. (T0, T and A1) were placed after treatment, in buffered formol for 24 h.

After 24 h, the samples were dehydrated and impregnated with paraffin using a Leica TP 1020 automatic dehydrating machine according to the operating procedure MO-H-149.

They were placed in a unit according to the operating procedure MO-H-153 using a Leica EG 1160 coating station.

5-μm sections were carried out according to the operating procedure MO-H-173 using a microtome of the Minot, Leica RM 2125 type and mounted on Superfrost® histological glass slides.

The microscopic observations were carried out with optical microscopy, using a Leica microscope of the Orthoplan or Olympus BX43 type. The images were taken with an Olympus DP72 camera and the CellD software.

The purpose of this analysis of images is to evaluate the variation in density and/or in thickness of the epidermis and/or of the papillary dermis initiated by the penetration of hydrophobised hyaluronic acid applied on the explants.

The results are presented in Table 5.

| Variation vs. T (D10) | Lot A1 |
|---|---|
| Epidermis | +293%** |
| Papillary dermis | +57%** |

**significant with p < 0.1 (99%).

The results show that after 10 days, the hyaluronic acid is substantially increased by 293% in the epidermis and by 57% in the papillary dermis compared to the untreated control lot (T) when the explant is treated with particles comprising O-palmitoyl-hyaluronic acid.

In conclusion, these results show that the hydrophobisation of hyaluronic acid makes it possible 1) to improve the penetration of the hyaluronic acid in the epidermis and the dermis regardless of the molar weight thereof and 2) makes it possible to increase the thickness of the skin structures. The formulations containing hydrophobised hyaluronic acid of the invention, in particular particles of O-palmitoyl-hyaluronic acid, can therefore be used to reduce the imperfections of the skin (for example, for filling in wrinkled and/or lines).

4.2. Particles of O -Stearyl Hyaluronic Acid AH-4-AS

An analysis of images was also carried out on the explants treated topically by the particles of O-stearyl hyaluronic acid (cf. example 3.2). The CellD software was used to process the images.

The results showed a substantial increase in the production of hyaluronic acid in the epidermis (7%) and in the dermis (68%) compared to the control lot (not treated).

In conclusion, the particles of the invention make it possible to stimulate the endogenous production of hyaluronic acid both in the epidermis and in the dermis.

### Example 5

### Healing Effect of Superficial Wounds

The purpose is to evaluate the action of particles of O-palmitoyl-hyaluronic acid AH-4-AP on the healing of superficial burns using skin explants ex-vivo.

A comparison was made between:
the particles of O-palmitoyl-hyaluronic acid obtained according to the protocol of example 2 (formulation A4); and
a commercial product (Cicalfate®, AVENE) known as a repairing cream for superficial injuries of the skin.

30 explants with a diameter of approximately 11 mm (±1 mm) were prepared using an abdominoplasty from a woman aged 31 years of the Caucasian type (reference P1496-AB31). The explants were maintained in a BEM medium (BIO-EC's Explants Medium) at 37° C. in a wet atmosphere, enriched with 5% CO₂.

At D0, each explant was placed in an HBSS medium (Hank's Balanced Saline Solution and irradiated at the centre with a dose of 10 J/cm² of UVB rays by using a Vilbert Lourmat RMX 3W solar stimulator.

The formulations were applied topically at a rate of 2 mg/cm² of product (spread with a small spatula) on the explants at days D0 (after irradiation), D2, D3, D6 and D8. The explants of lots T and B did not receive any treatment other than the renewing of the medium.

Table 6 shows the various lots tested:

| | | | Number | |
|---|---|---|---|---|
| Lot | Treatment | Burn | of explants tested | Treatment stopped |
| T0 | None | None | 3 | D0 |
| T | None | None | 6 | D3 and D10 |
| B | None | Yes | 9 | D3 and D10 |
| BR | Cicalfate ® | Yes | 6 | D3 and D10 |
| A4 | AH-4-AP | Yes | 6 | D3 and D10 |

At D0, the explants of the lot T0 were sampled and separated into two. A half-explant was fixed in buffered formol and the other was frozen at −80° C. At D3 and D10, the explants were sampled and treated in the same way.

After 24 h of fixing in the buffered formol, the samples were dehydrated and impregnated with paraffin using a Leica TP 1020 automatic dehydrating machine. They were placed in a unit using a Leica EG 1160 coating station. 5-μm sections were carried out using a microtome of the Minot, Leica RM 2125 type and mounted on Superfrost® histological glass slides.

The microscopic observations were carried out with optical microscopy, using a Leica microscope of the DMLB or Olympus BX43 type. The images were taken with an Olympus DP72 camera and the CellD software.

The results of the analysis of the images show that the particles of hydrophobised hyaluronic acid have a healing activity of which the effectiveness is comparable to the commercial product Cicalfate®.

Consequently, the particles of hydrophobised hyaluronic acid presents an interesting alternative to the products currently available in the market.

1-12. (canceled)

13. A cosmetic agent comprising particles comprising hyaluronic acid modified by hydrophobic groups or the salts and/or derivatives thereof, having the formula (I): 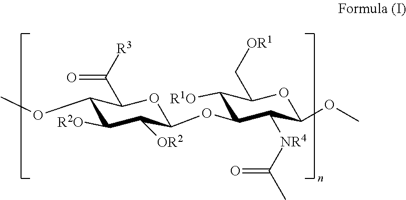
wherein
R¹ and R² each independently represent a hydrogen atom, an R group or a —COR group wherein R represents:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
R³ represents:
a hydroxyl group;
a —O⁻X⁺ group wherein X⁺ represents a cation; preferably, a sodium ion; or
a —OR′ group or a —NHR′ group wherein R′ represents:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
R⁴ represents a hydrogen atom or an R″ group chosen from:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group
with said particles not comprising cyclodextrin;
said hyaluronic acid modified by the hydrophobic groups or its salts, has a degree of substitution within a range from 0.2 to 5%.

14. The cosmetic agent according to claim 13 wherein the particles are particles of O-palmitoyl-hyaluronic acid, of O-oleoyl-hyaluronic acid, of O-stearyl-hyaluronic acid or of O-lauryl-hyaluronic acid.

15. The cosmetic agent according to claim 13, as a volumising agent and/or plumping agent.

16. The cosmetic agent according to claim 13, in order to improve the moisturising of the mucous members, of the epidermis and of the dermis.

17. The cosmetic agent according to claim 13, for limiting and/or correcting imperfections of the skin; preferably, as a tensing agent of the skin or as an agent that favours the filling in of wrinkles and/or lines of the skin.

18. Particles comprising hyaluronic acid modified by hydrophobic groups, or the salts thereof, having the formula (I): 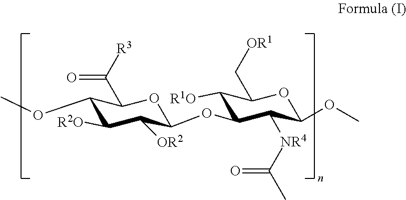
wherein
R¹ and R² each independently represent a hydrogen atom, an R group or a —COR group wherein R represents:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
R³ represents:
a hydroxyl group;
a —O⁻X⁺ group wherein X⁺ represents a cation; preferably, a sodium ion; or
a —OR′ group or a —NHR′ group wherein R′ represents:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
R⁴ represents a hydrogen atom or an R″ group chosen from:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group;
with said particles not comprising cyclodextrin;
said hyaluronic acid modified by the hydrophobic groups or its salts, has a degree of substitution within a range from 0.2 to 5%;
for use as a medicament.

19. Particles of hyaluronic acid for their use as a medicament according to claim 18, wherein the particles are used as a dermatological agent; preferably, as a healing agent.

20. Particles comprising hyaluronic acid modified by hydrophobic groups, or the salts thereof, having the formula (I): 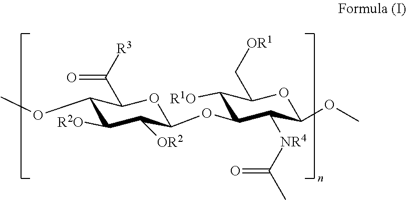
wherein
R¹ and R² each independently represent a hydrogen atom, an R group or a —COR group wherein R represents:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
R³ represents:
a hydroxyl group;
a —O⁻X⁺ group wherein X⁺ represents a cation; preferably, a sodium ion; or
a —OR′ group or a —NHR′ group wherein R′ represents:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
R⁴ represents a hydrogen atom or an R″ group chosen from:
a linear or branched alkyl group, containing from 1 to 1,000 carbon atoms; preferably, containing from 1 to 60 carbon atoms; more preferably, containing from 1 to 20 carbon atoms; more preferably, R represents a linear alkyl group containing 15 or 16 carbon atoms; or
a linear or branched alkene group, containing 2 to 1,000 carbon atoms and bearing at least one C═C double bond;
n represents the number of disaccharide units of D-N-glucuronic acid and of N-acetyl glucosamine and represents a strictly positive integer;
with the condition that at least one of R¹ or R² represents a —R or —COR group; or R³ represents a —OR′ group or a —NHR′ group; or R⁴ represents an R″ group;
with said particles not comprising cyclodextrin;
said hyaluronic acid modified by the hydrophobic groups or its salts, has a degree of substitution within a range from 0.2 to 5%.

21. Particles according to claim 20, comprising hyaluronic acid modified by hydrophobic groups, of the salts and/or derivatives thereof, having the formula (Ia): 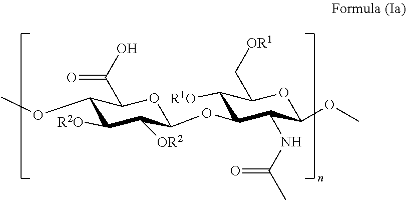
wherein at least one of R¹ or R² represents a —COR group.

22. Pharmaceutical composition comprising particles according to claim 20 and a pharmaceutically acceptable carrier.

23. Medical device comprising particles according to claim 20.

24. Medical device according to claim 23 for use in repairing superficial lesions of the skin or of the mucous membranes; preferably for use in healing superficial wounds of the skin.
